(19) United States
(12) Patent Application Publication
(10) Pub. No.: US 2024/0115527 A1
(43) Pub. Date: Apr. 11, 2024

(54) CXCR1/CXCR2 INHIBITORS FOR USE IN TREATING MYELOFIBROSIS

(71) Applicant: Icahn School of Medicine at Mount Sinai, New York, NY (US)

(72) Inventors: Ronald Hoffman, New York, NY (US); Min Lu, New York, NY (US)

(73) Assignee: Icahn School of Medicine at Mount Sinai, New York, NY (US)

(21) Appl. No.: 18/251,670

(22) PCT Filed: Nov. 4, 2021
(86) PCT No.: PCT/US21/57986

§ 371 Date: May 3, 2023

Related U.S. Application Data
(60) Provisional application No. 63/109,981, filed on Nov. 5, 2020.

(51) Int. Cl. A61K 31/18; A61P 19/08

(52) U.S. Cl. CPC A61K 31/18; A61P 19/08

(57) ABSTRACT

Provided herein are compositions comprising CXCR1/CXCR2 inhibitors as well as methods of using the CXCR1/CXCR2 inhibitors disclosed herein. In embodiments, provided are methods of treating myelofibrosis, methods of decreasing bone marrow fibrosis, methods of reducing the interaction of IL-8 to CXCR1 and/or CXCR2, and methods of reducing the activity or and/or signaling through CXCR1 and/or CXCR by administering to a subject in need thereof an effective amount of a CXCR1/CXCR2 inhibitor disclosed herein.

### CROSS-REFERENCE AND CLAIM OF PRIORITY

This application is a national phase of International Patent Application No. PCT/US2021/57986 filed Nov. 4, 2021, and which claims the benefit of U.S. Provisional Application No. 63/109,981 filed Nov. 5, 2020. The entirety of which are both incorporated herein by reference.

### STATEMENT REGARDING FEDERALLY SPONSORED RESEARCH

This invention was made with government support under Grant Number NIH 5P01CA108671-11 awarded by the National Institutes of Health. The government has certain rights in the invention.

### FIELD OF THE INVENTION

This disclosure relates to compositions and methods for treating myelofibrosis.

### BACKGROUND

Myelofibrosis (MF) is a myeloproliferative neoplasm that arises from clonal proliferation of malignant hematopoietic stem cells (HSC) and leads to progressive bone marrow (BM) fibrosis.

The clinical phenotype in both primary MF and post polycythemia vera/essential thrombocythemia myelofibrosis (post-PV/ET) MF is a consequence of both the primary clonal myeloproliferative neoplasm and a secondary inflammatory milieu that is characterized by bone marrow fibrosis, increased marrow micro-vessel density, osteosclerosis, and an aberrant cytokine milieu. Specifically, MF involves the constitutive mobilization of hematopoietic progenitor cells (HPC) and HSC with genetic abnormalities, including mutations that directly or indirectly induce upregulation of the JAK-STAT pathway. Further, tissue-specific microenvironments can create niches that favor the predominance of these malignant HSC/HPC at the expense of normal HSC/HPC. Various cytokines produced by the malignant hematopoietic cells act on bone marrow stromal cells to cause a proliferation of reactive polyclonal bone marrow stromal cells, which leads the fibrosis of bone marrow, osteosclerosis and angiogenesis. Finally, this results in characteristic clinical symptoms such as an ineffective hematopoiesis, an appearance of dacryocytes in peripheral blood, leukoerythroblastosis, systemic symptoms, and extramedullary hematopoiesis causing a splenomegaly.

MF patients inevitably develop increasing symptoms and marrow failure and have a 10-20% of risk of developing a form of acute myeloid leukemia (AML) that is refractory to chemotherapy and is associated with a median survival of 3-5 months. While allogeneic stem cell transplantation can be curative, it is not available to most MF patients.

Further, although myeloproliferative neoplasms (MPNs) are uniformly associated with the activation of the JAK/STAT signaling pathways, therapy with the competitive FDA approved JAK1/2 inhibitor, ruxolitinib, results in significant clinical benefits but a modest if any effect on survival. The limited benefits of reversible JAK1/2 inhibitor therapy are likely due to its inability to deplete or eliminate malignant hematopoietic stem/progenitor cell (HSC/HPC) numbers.

Accordingly, agents and methods to deplete malignant HSCs, allowing the recovery of a reservoir of normal HSCs, and to inhibit inflammatory signaling in MF are urgently needed.

### SUMMARY OF THE INVENTION

In one aspect, provided is a CXCR1/CXCR2 inhibitor for use in the treatment of myelofibrosis (MF) in a subject in need thereof.

In one aspect, provided is a CXCR1/CXCR2 inhibitor for use in decreasing bone marrow fibrosis, spleen volume, plasma VEGF levels, bone marrow microvessel density, bone marrow megakaryocyte number, number of IL-8 secreting clones, and/or number of peripheral blood CD34⁺ cells in a subject.

In one aspect, provided is a CXCR1/CXCR2 inhibitor for use in a method of reducing the interaction of IL-8 to an IL-8 receptor in a subject in need thereof.

In one aspect, provided is a CXCR1/CXCR2 inhibitor for use in a method of reducing the activity or and/or signaling through an IL-8 receptor in a subject in need thereof.

In one aspect, provided is a CXCR1/CXCR2 inhibitor for use in reducing IL-8 signaling in a subject in need thereof.

In one aspect, provided is a method of treating MF, the method comprising administering to a subject in need thereof an effective amount of a CXCR1/CXCR2 inhibitor.

In one aspect, provided is a method of decreasing bone marrow fibrosis, spleen volume, plasma VEGF levels, bone marrow microvessel density, bone marrow megakaryocyte number, number of IL-8 secreting clones, and/or number of peripheral blood CD34⁺ cells in a subject, the method comprising administering to a subject in need thereof an effective amount of a CXCR1/CXCR2 inhibitor.

In one aspect, provided is a method of reducing the interaction of IL-8 to CXCR1 and/or CXCR2, the method comprising administering to a subject in need thereof an effective amount of a CXCR1/CXCR2 inhibitor.

In one aspect, provided is a method of reducing the activity or and/or signaling through CXCR1 and/or CXCR, the method comprising administering to a subject in need thereof an effective amount of a CXCR1/CXCR2 inhibitor.

In one aspect, provided is a method of reducing IL-8 signaling, the method comprising administering to a subject in need thereof an effective amount of a CXCR1/CXCR2 inhibitor.

In some embodiments, the subject is unresponsive to or ineligible for janus kinase inhibitor (JAKi) treatment.

In one embodiment, the subject has MF.

In some embodiments, the CXCR1/CXCR2 inhibitor is administered as a pharmaceutical composition comprising the CXCR1/CXCR2 inhibitor and one or more pharmaceutically acceptable excipients.

In some embodiments, the CXCR1/CXCR2 inhibitor is a compound of formula (I)

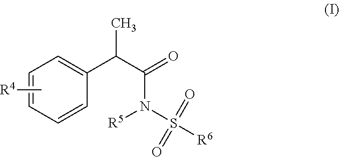

or a pharmaceutically acceptable salt thereof,
wherein
R⁴ is linear or branched C₁-C₆ alkyl, benzoyl, phenoxy, trifluoromethanesulfonyloxy; preferably it is selected from benzoyl, isobutyl and trifluoromethanesulfonyloxy. Also, according to a preferred embodiment R⁴ is in position 3 or 4 on the phenyl ring, more preferably it is 3-benzoyl, 4-isobutyl or 4-trifluoromethanesulfonyloxy.
R⁵ is H or linear or branched C₁-C₃ alkyl, preferably it is H.
R⁶ is linear or branched C₁-C₆ alkyl or halo C₁-C₃ alkyl, preferably it is CH₃ or trifluoromethyl.

In some embodiments, in the CXCR1/CXCR2 inhibitor of formula (I):
R⁴ is C₁-C₆ alkyl or benzoyl; preferably it is in positions 3 and 4, more preferably, it is 3-benzoyl or 4-isobutyl.
R⁵ is H or linear or branched C₁-C₃ alkyl, preferably it is H,
R⁶ is linear or branched C₁-C₆ alkyl or trifluormethyl; preferably it is a linear or branched C₁-C₆ alkyl, more preferably it is CH₃.

In some embodiments, in the CXCR1/CXCR2 inhibitor of formula (I):
R4 is trifluoromethanesulfonyloxy, preferably 4-trifluoromethanesulfonyloxy,
R5 is H or linear or branched C1-C3 alkyl, preferably it is H,
R6 is linear or branched C1-C6 alkyl or trifluormethyl; preferably it is a linear or branched C1-C16 alkyl, moire preferably it is CH3.

In some embodiments, the CXCR1/CXCR2 inhibitor is a small molecule of formula (II):

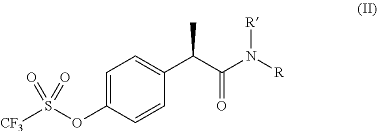

or a pharmaceutically acceptable salts thereof,
wherein
R′ is hydrogen;
R is a residue of formula SO2Ra wherein Ra is C₁-C₆ alkyl or halo C₁-C₃ alkyl, preferably it is CH₃ or trifluoromethyl.

In some embodiments, the asymmetric carbon substituted with methyl in formulas (I) and (II) has absolute configuration R.

In some embodiments, the CXCR1/CXCR2 inhibitor is R(-)-2-[(4′-trifluoromethanesulfonyloxy)phenyl]-N-methanesulfonyl propionamide or its sodium salt ((also known as ladarixin or DF2156A).

In one aspect, provided is ladaxirin for use in the treatment of MF in a subject in need thereof.

In one aspect, provided is a method of treating MF, the method comprising administering to a subject in need thereof an effective amount of ladarixin.

In some embodiments, the CXCR1/CXCR2 inhibitor is a small molecule of formula (V)

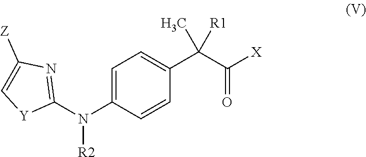

wherein
R1 is hydrogen;

### X is OH;

R2 is hydrogen or linear C₁-C₄ alkyl;
Y is a heteroatom selected from S, O and N;
Z is selected from linear or branched C₁-C₄ alkyl, linear or branched C₁-C₄ alkoxy, halo C₁-C₃ alkyl and halo C₁-C₃ alkoxy.

In one embodiment, the CXCR1/CXCR2 inhibitor is R-(-)-2-(4-isobutylphenyl)-N-methanesulfonyl propionamide or its lysine salt (also known as reparixin).

In one aspect, provided is reparixin for use in the treatment of MF in a subject in need thereof.

In one aspect, provided is a method of treating MF, the method comprising administering to a subject in need thereof an effective amount of reparixin.

### BRIEF DESCRIPTION OF THE FIGURES

FIGS. 1A, 1B, 1C, and 1D illustrate the important role that IL-8 plays in MF disease development. FIG. 1A shows that IL-8 levels are higher in MF plasma as compared to plasma from normal, polycythemia vera (PV) patients, essential thrombocythemia (ET) patients, or healthy individuals. FIG. 1B illustrates that MF patients with expanded IL-8 secreting clones (defined as >50% of total CD34⁺ cells) had also increased leukocytosis (p<0.0001), larger spleen sizes (p=0.0004), greater prevalence of constitutional symptoms (p=0.0084), and higher-grade reticulin fibrosis in marrow in comparison to MF patients without prevalent IL-8 clones. MF IL-8 <50%: 24% Grade 0; 52% Grade 1; 16% Grade 2; 8% Grade 3. MF IL-8 >50%: 12% Grade 1; 38% Grade 2, 50% Grade 3. FIG. 1C illustrates immunohistochemistry (IHC) experiments confirming increased IL-8 expression in marrow biopsies from 8/15 MF patients in comparison to 0/4 normal controls. FIG. 1D illustrates that high IL-8 expression was observed in MF splenic megakaryocytes (MKs) as well as in splenic stromal/endothelial cells not seen in normal spleen.

FIGS. 2A, 2B, 2C, 2D, and 2E illustrate that IL-8 receptors CXCR1/CXCR2 play an important role in MF. FIG. 2A illustrates that normal and MF splenic tissues express CXCR1 (A) and CXCR2 (B) in littoral cells. FIG. 2B illustrates that MF splenic samples contained higher percentage of CD34⁺/CXCR1⁺ and CD34⁺/CXCR2⁺ cells than MF peripheral blood (PB) samples. FIG. 2C shows that IL-8-high MF CD34⁺ cells have enhanced surface expression of CXCR2 and its analog CXCR1 as compared to normal cells, such that MF was characterized by increased IL-8 ligand and receptor expression. pts=patients. FIG. 2D shows that enhanced surface expression of CXCR1/CXCR2 coincided with enhanced NFkB pathway activity. FIG. 2E shows that as determined by FACS, a higher fraction of JAK2V617F positive MF CD34⁺ cells than normal CD34⁺ cells expressed CXCR1 and CXCR2 receptors (p=0.01 and p=0.006, respectively).

FIGS. 3A, 3B, and 3C illustrate that reduction of IL-8 blocks VEGF, which is involved in the development of splenic endothelial cells (EC)/MF HSC niches. FIGS. 3A and 3B illustrate that LCN2 increases IL-8 and CXCL1 protein and mRNA levels in spleen stromal cells. Con: left bars. LCN2: right bars. FIG. 3C illustrates that IL-8 regulates VEGF expression. si=silencing. Ab=antibody. Con=control.

FIGS. 4A and 4B illustrate that addition of a CXCR1/CXCR2 inhibitor reverses effects of IL-8 on MF CD34⁺ cells proliferation and lineage differentiation. FIG. 4A illustrates that IL-8 decreased the fraction of normal CD34⁺, CD41⁺, and CD33⁺ cells, but increased the fraction of MF CD34⁺, CD41⁺, and CD33⁺ cells. The effects of IL-8 were eliminated by the addition of the CXCR1/CXCR2 inhibitor ladarixin (Ladx). Normal CD34/CD41/CD33: left bars. MF CD34/CD41/CD33: Right bars. FIG. 4B illustrates that splenic endothelial cells (ECs) promote the proliferation of hematopoietic CD34⁺ cells. Shown is fold change in CD34⁺ cells cultured alone in the absence of cytokines and that the numbers are increased when the CD34⁺ cells were co-cultured with LCN2 treated endothelial cells. The effects of co-cultivation with ECs were eliminated by addition of the CXCR1/CXCR2 antagonist reparixin (RPX).

FIGS. 5A, 5B. 5C, 5D, and 5E illustrate that a CXCR1/CXCR2 inhibitor reverses effects of IL-8 on MF CD34⁺ cell colony formation. FIG. 5A shows colony forming assays of cultured MF CD34⁺ cells, demonstrating enhanced colony output when cultured with IL-8 compared to WT CD34⁺ cells—an effect ameliorated by co-treatment with the CXCR1/CXCR2 inhibitor RPX. CFU-GM=colony-forming unit-granulocyte-macrophage. FIGS. 5B and 5C illustrate that IL-8 increased CFU-GM colony formation by MF CD34⁺ cells in a dose dependent fashion and that the effects of IL-8 were inhibited by treatment with Ladx. FIG. 5B. Normal cells. FIG. 5C. MF cells. FIG. 5D shows that treatment with IL-8 increased CFU-GM colony formation in MF samples with the highest expression of CXCR1/CXCR2. Con=control. IL-8 concentrations increase from left to right. FIG. 5E shows that these effects could be mitigated by addition of CXCR1/CXCR2 inhibitor Ladx. P values were as follows: w/o Ladx: Con vs IL-8—10 ng: 0.159332; Con vs IL-8—20 ng: 0.011976; Con vs IL-8—50 ng: 0.00262; Con vs IL-8—100 ng: 0.00042; w/ 10 μM Ladx: Con vs Ladx: 0.315782; IL-8—10 ng vs plus Ladx: 0.295851; IL-8—20 ng vs plus Ladx: 0.077726; IL-8—50 ng vs plus Ladx: 0.031355; IL-8—100 ng vs plus Ladx: 0.081595.

FIGS. 6A, 6B, 6C, 6D, and 6E illustrate that a CXCR1/CXCR2 inhibitor reverses effects of IL-8 on micro-environmental cells. FIG. 6A shows the effects of MF hematopoietic cells on the morphology of splenic adherent cells after co-cultivation for three days. N splenic AC=normal splenic adherent cells. nBM MNC=non-adherent bone marrow mononuclear cells. MNC=mononuclear cells. FIGS. 6B and 6C show that stromal cells and MNC cells individually produced less IL-8 (FIG. 6B) and VEGF (FIG. 6C) as compared to co-cultured stromal cells and MNC cells. FIGS. 6D and 6E illustrate that addition of CXCR1/CXCR2 inhibitor Ladx decreased the levels of IL-8 (FIG. 6D) and VEGF (FIG. 6E) in conditioned medium harvested from co-cultures of MF MNC and stromal cells.

### DETAILED DESCRIPTION OF THE INVENTION

Provided herein are CXCR1/CXCR2 inhibitors for use in the treatment of myelofibrosis (MF). Provided herein are methods of using a CXCR1/CXCR2 inhibitor for the treatment of MF.

CXCR1 and CXCR2 are receptors for the cytokine IL-8. As used herein, the term “CXCR1/CXCR2 inhibitor” refers to any compound able to inhibit, partially or totally, signaling through CXCR1 or through CXCR1 and CXCR2 and/or able to inhibit, partially or totally, the interaction of IL-8 with the CXCR1 or with the CXCR1 and CXCR2 receptors. In some embodiments, the CXCR1/CXCR2 inhibitor inhibits both CXCR1 and CXCR2.

In some embodiments, the CXCR1/CXCR2 inhibitor is ladarixin or a ladarixin derivative.

In one embodiment, the CXCR1/CXCR2 inhibitor is a small molecule of formula (I)

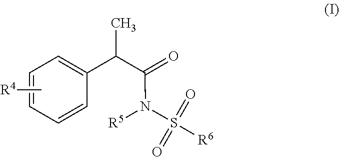

or a pharmaceutically acceptable salt thereof,
wherein
R⁴ is linear or branched C₁-C₆ alkyl, benzoyl, phenoxy, trifluoromethanesulfonyloxy; preferably it is selected from benzoyl, isobutyl and trifluoromethanesulfonyloxy. Also, according to a preferred embodiment R⁴ is in position 3 or 4 on the phenyl ring, more preferably it is 3-benzoyl, 4-isobutyl or 4-trifluoromethanesulfonyloxy.
R⁵ is H or linear or branched C₁-C₃ alkyl, preferably it is H.
R⁶ is linear or branched C₁-C₆ alkyl or halo C₁-C₃ alkyl, preferably it is CH₃ or trifluoromethyl.

In some embodiments, in the CXCR1/CXCR2 inhibitor of formula (I):
R⁴ is C₁-C₆ alkyl or benzoyl; preferably it is in positions 3 and 4, more preferably, it is 3-benzoyl or 4-isobutyl.
R⁵ is H or linear or branched C₁-C₃ alkyl, preferably it is H,
R⁶ is linear or branched C₁-C₆ alkyl or trifluormethyl; preferably it is a linear or branched C₁-C₆ alkyl, more preferably it is CH₃.

In some embodiments, in the CXCR1/CXCR2 inhibitor of formula (I):
R4 is trifluoromethanesulfonyloxy, preferably 4-trifluoromethanesulfonyloxy,
R5 is H or linear or branched C1-C3 alkyl, preferably it is H,
R6 is linear or branched C1-C6 alkyl or trifluormethyl; preferably it is a linear or branched C1-C16 alkyl, moire preferably it is CH3.

In some embodiments, the CXCR1/CXCR2 inhibitor is a small molecule of formula (II):

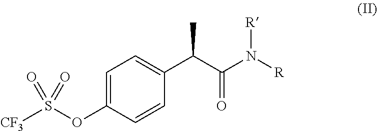

or a pharmaceutically acceptable salts thereof,
wherein
R′ is hydrogen;
R is a residue of formula SO₂Ra wherein Ra is C₁-C₆ alkyl or halo C₁-C₃ alkyl, preferably it is CH₃ or trifluoromethyl.

In some embodiments, the asymmetric carbon substituted with methyl in formulas (I) and (II) has absolute configuration R.

In some embodiments, the CXCR1/2 is R-(-)-2-(4-isobutylphenyl)propionyl methansulfonamide and pharmaceutically acceptable salts thereof.

In some embodiments, the CXCR1/2 is the lysine salt of R-(-)-2-(4-isobutylphenyl)propionyl methansulfonamide (also known as Reparixin).

In some embodiments, the CXCR1/2 is R-(-)-2-(4-trifluoromethanesulfonyloxy)phenyl[-N-methanesulfonyl propionamide. In some embodiments, the CXCR1/2 is the sodium salt of R-(-)-2-(4-trifluoromethanesulfonyloxy)phenyl]-N-methanesulfonyl propionamide (also known as Ladarixin).

In some embodiments, the CXCR1/CXCR2 inhibitor is R(-)-2-[(4′-trifluoromethanesulfonyloxy)phenyl]-N-methanesulfonyl propionamide (also known as DF2156Y) and its sodium salt (also known as Ladarixin or DF2156A).

In one embodiment, the CXCR1/CXCR2 inhibitor has formula III:

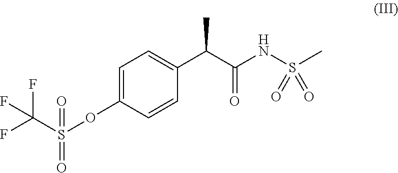 [SMILES: C[C@@H](C(=O)NS(C)(=O)=O)c1ccc(OS(=O)(=O)C(F)(F)F)cc1]

In some embodiments, the CXCR1/CXCR2 inhibitor is a sodium salt of the small molecule of formula III (ladarixin, CAS No.: 865625-56-5).

In some embodiments, the CXCR1/CXCR2 inhibitor is a small molecule disclosed in PCT application publication number WO2005/090295, which is hereby incorporated in its entirety.

In some embodiments, the CXCR1/CXCR2 inhibitor is reparixin or a reparixin derivative.

In one embodiment, the CXCR1/CXCR2 inhibitor has formula VI:

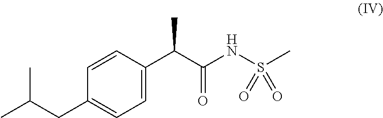 [SMILES: CC(C)Cc1ccc([C@@H](C)C(=O)NS(C)(=O)=O)cc1]

In some embodiments, the CXCR1/CXCR2 inhibitor is a L-lysine salt of the small molecule of formula IV (reparixin, CAS No. 266359-93-7).

In some embodiments, the CXCR1/CXCR2 inhibitor is a small molecules disclosed in PCT application WO2000/024710, which is hereby incorporated by reference in its entirety.

In some embodiments, the CXCR1/CXCR2 inhibitor is a small molecule of formula (V)

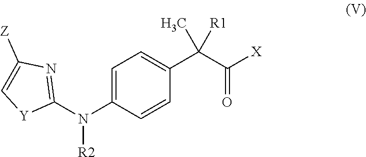

wherein
R1 is hydrogen;

### X is OH;

R2 is hydrogen or linear C₁-C₄ alkyl;
Y is a heteroatom selected from S, O and N;
Z is selected from linear or branched C₁-C₄ alkyl, linear or branched C₁-C₄ alkoxy, halo C₁-C₃ alkyl and halo C₁-C₃ alkoxy.

In some embodiments, the CXCR1/CXCR2 inhibitor is a small molecules disclosed in PCT application WO2010/031835, which is hereby incorporated by reference in its entirety.

More preferably, said compounds of formula (V) have the chiral carbon atom of the phenylproprionic group in the S configuration.

In some embodiments, the CXCR1/CXCR2 inhibitor is (2S)-2-(4-{[4-(trifluoromethyl)-1,3-thiazol-2-yl] amino} phenyl) propanoic acid and pharmaceutically acceptable salts thereof, preferably its sodium salt.

In some embodiments, the CXCR1/CXCR2 inhibitor is 2-methyl-2(4-{[4-(trifluoromethyl)-1,3-thiazol-2-yl] amino} phenyl) propanoic acid and pharmaceutically acceptable salts thereof, preferably its sodium salt.

Provided herein is a CXCR1/CXCR2 inhibitor as disclosed hereinabove for use in the treatment, the prevention of, and/or reducing the likelihood of developing MF in a subject in need thereof. Provided herein are methods and compositions for the treatment, for the prevention of, and/or for reducing the likelihood of developing MF, the methods comprising administering to a subject in need thereof an effective amount of an CXCR1/CXCR2 inhibitor.

The administration of the CXCR1/CXCR2 inhibitor can occur before, during, or after a diagnosis of MF has been made.

By “subject” is meant a mammal, including, but not limited to, a human or non-human mammal. The mammal may be a commercially farmed animal (such as a horse, a cow, a sheep or a pig), a laboratory animal (such as a mouse or a rat), or a pet (such as a cat, a dog, a rabbit or a guinea pig). The subject is preferably a human. The subject may be male or female. Individuals and patients are also subjects herein.

The terms “treat,” “treated,” “treating,” or “treatment” as used herein refer to a therapeutic treatment, wherein the object is to slow down (lessen) an undesired physiological condition, disorder or disease, or to obtain beneficial or desired clinical results. For the purposes of this disclosure, beneficial or desired clinical results include, but are not limited to, alleviation of symptoms, diminishment of the extent of the condition, disorder or disease, stabilization (i.e., not worsening) of the state of the condition, disorder or disease, slowing of the progression of the condition, disorder or disease, amelioration of the condition, disorder or disease state, remission (whether partial or total), whether detectable or undetectable, or enhancement or improvement of the condition, disorder or disease. Treatment includes eliciting a clinically significant response without excessive levels of side effects. Treatment also includes prolonging survival as compared to expected survival if not receiving treatment. In some embodiments, the treatment results in a reduction of MF symptoms, including, but not limited to anemia, weakness, fatigue, bleeding, abnormally enlarged spleen (splenomegaly), and pain.

In some embodiments, the disclosure provides therapeutic methods, wherein a therapeutically effective amount of an CXCR1/CXCR2 inhibitor is administered to a subject in need thereof. “Therapeutically effective amount” means an amount of an antibody or antigen-binding fragment thereof set forth herein that, when administered to a subject, is effective in producing the desired therapeutic effect. A therapeutically effective amount may also refer to a combination of more than one CXCR1/CXCR2 inhibitor, which in combination lead to the desired therapeutic effect. Therapeutic effects include a clinical improvement by International Working Group-Myeloproliferative Neoplasms Research and Treatment (IWG-MRT) and/or European LeukemiaNet (ELN) criteria, a decrease in bone marrow fibrosis, a reduction in spleen volume, reduced plasma VEGF level, reduced bone marrow microvessel density, decreased bone marrow fibrosis grade, reduced bone marrow megakaryocyte number, reduced number of IL-8 secreting clones, and reduced number of peripheral blood CD34⁺ cells.

The patient may be asymptomatic and/or may have a predisposition to the disease. As such, in one embodiment the disclosure provides methods of reducing the likelihood, delaying, or preventing the onset of developing MF. The disclosure also provides prophylactic methods, wherein a prophylactically effective amount of a CXCR1/CXCR2 inhibitor is be administered to a subject in need thereof. A “prophylactically effective amount” is an amount that prevents, reduces, and/or delays the onset of one or more symptoms of the disease. A prophylactically effective amount may also refer to a combination of more than one CXCR1/CXCR2 inhibitor which in combination leads to the desired prophylactic effect. Prophylactic and preventive are used interchangeably herein.

Provided is a CXCR1/CXCR2 inhibitor for use in a method of reducing the interaction of IL-8 to CXCR1 and/or CXCR2 in a subject in need thereof.

Provided is a CXCR1/CXCR2 inhibitor for use in a method of reducing the activity or and/or signaling through CXCR1 and/or CXCR2 in a subject in need thereof.

Provided is a CXCR1/CXCR2 inhibitor for use in a reducing IL-8 signaling in a subject in need thereof.

Provided is a method of reducing the interaction of IL-8 to CXCR1 and/or CXCR2, the method comprising administering to a subject in need thereof an effective amount of a CXCR1/CXCR2 inhibitor.

Provided is a method of reducing the activity or and/or signaling through CXCR1 and/or CXCR2, the method comprising administering to a subject in need thereof an effective amount of a CXCR1/CXCR2 inhibitor.

Provided is a method of reducing IL-8 signaling, the method comprising administering to a subject in need thereof an effective amount of a CXCR1/CXCR2 inhibitor.

In some embodiments, the subject has previously received janus kinase inhibitor (JAKi) therapy. In some embodiments, the subject has previously received JAKi therapy and is now unresponsive to JAKi therapy. Lack of responsiveness to JAKi therapy may be found, for example, by when (1) a subject is treated with JAKi therapy for ≥3 months with an inadequate efficacy response defined as <10% spleen volume reduction by MRI or <30% decrease from baseline in spleen length by physical examination or regrowth to these parameters following an initial response; and/or (2) a treatment for ≥28 days complicated by the development of a red blood cell transfusion requirement or thrombocytopenia, anemia, hematoma, and/or hemorrhage occur during treatment.

As used herein, the term “administration” refers to a drug to a physiological system (e.g., subject or in vivo, in vitro, or ex vivo cells, tissues, and organs), or refers to the act of giving therapeutic treatment. Typical routes of administration to the human body are the eye (ocular), mouth (oral), skin (transdermal), nose (nasal), lung (inhaled antigen), oral mucosa (in the cheek), Through the ear, injection (e.g., intravenous, subcutaneous, intratumor, intraperitoneal, etc.) and similar methods can be used. A preferred route of administration according to the present invention is oral administration. A preferred route of administration according to the present invention is oral administration.

Depending on the intended route of delivery, the compounds are preferably formulated as either injectable or oral compositions. The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, the acid compound is usually a minor component from about 0.1 to about 50% by weight or preferably from about 1 to about 40% by weight) with the remainder being various vehicles or carriers and processing aids helpful for forming the desired dosing form. Liquid forms suitable for oral administration may include a suitable aqueous or non-aqueous vehicle with buffers, suspending and dispensing agents, colorants, flavors and the like. Liquid forms, including the injectable compositions described here below, are usually stored in the absence of light, so as to avoid any catalytic effect of light, such as hydroperoxide or peroxide formation. In the methods disclosed herein, the CXCR1/CXCR2 inhibitor may be administered in a pharmaceutically acceptable compositions that comprises the CXCR1/CXCR2 inhibitor formulated together with one or more pharmaceutically acceptable excipients. A pharmaceutically acceptable excipient can be a pharmaceutically-acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting the therapeutic compound for administration to the subject, bulking agent, salt, surfactant and/or a preservative. Some examples of materials which can serve as pharmaceutically-acceptable excipients include: sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations.

The dosage of the CXCR1/CXCR2 inhibitor administered to the subject may vary, depending on specific inhibitor used, the reason for use, the individual subject, and the mode of administration. The dosage may be adjusted based on the subject's weight, sex, age and health of the subject, and tolerance for the CXCR1/CXCR2 inhibitor.

A dose of the CXCR1/CXCR2 inhibitor may be about 1 to about 1500 mg. A dose dosage of the CXCR1/CXCR2 inhibitor may be about 100 to about 1000 mg. A dose of the CXCR1/CXCR2 inhibitor may be about 100 mg, about 200 mg, about 300 mg, about 400, mg, about 500 mg, about 600 mg, about 700 mg, about 800 mg, about 900 mg, about 1000, about 1100 mg, about 1200 ng, about 1300 mg, about 1400 mg, or about 1500 mg. A daily dose of the CXCR1/CXCR2 inhibitor may be about 100 mg, about 200 mg, about 300 mg, about 400, mg, about 500 mg, about 600 mg, about 700 mg, about 800 mg, about 900 mg, about 1000, about 1100 mg, about 1200 mg, about 1300 mg, about 1400 mg, or about 1500 mg. In some embodiments, the dose of the CXCR1/CXCR2 inhibitor is 1200 mg.

Daily doses may be given in divided doses 1 to 5 times a day by oral administration or given by continuous infusion for 1 or more cycles of 5 to 10 days are effective to obtain desired results. Second or subsequent administrations can be at a dosage which is the same, less than or greater than the initial or previous dose administered to the individual. In certain embodiments, a dose of the CXCR1/CXCR2 inhibitor is administered to a subject every day, every other day, every couple of days, every third day, once a week, twice a week, three times a week, once every two weeks, or once a month.

In some embodiments, a dose(s) of a compound or a composition is administered for 2 days, 3 days, 5 days, 7 days, 14 days, 21 days or 28 days. In certain embodiments, a dose of a compound or a composition is administered for 1 month, 1.5 months, 2 months, 2.5 months, 3 months, 4 months, 5 months, 6 months or more.

In some embodiments, the CXCR1/CXCR2 inhibitor is reparixin and may be administered at 1200 mg, three times a day for cycles of 28 consecutive days. In some embodiments, the CXCR1/CXCR2 inhibitor is administered up to 24 weeks of treatment with the possibility to continue in case of benefit.

In some embodiments, the CXCR1/CXCR2 inhibitor is ladarixin and may be administered at 400 mg, two times a day for cycles of 14 consecutive days. In some embodiments, the CXCR1/CXCR2 inhibitor is administered for 3 cycles of 14 days on and 14 days off of treatment with the possibility to continue in case of benefit.

Provided is a method of reducing the interaction of IL-8 to CXCR1/CXCR2, the method comprising contacting a cell expressing CXCR1 and/or CXCR2 with a CXCR1/CXCR2 inhibitor.

Provided is a method of reducing the activity or and/or signaling through CXCR1/CXCR2, the method comprising contacting a cell expressing CXCR1 and/or CXCR2 with a CXCR1/CXCR2 inhibitor.

Provided is a method of reducing IL-8 signaling, the method comprising contacting a cell expressing CXCR1 and/or CXCR2 with a CXCR1/CXCR2 inhibitor.

### EXAMPLES

### Example 1: The Pro-Inflammatory Cytokine IL-8 is Increased in Patients with Myelofibrosis

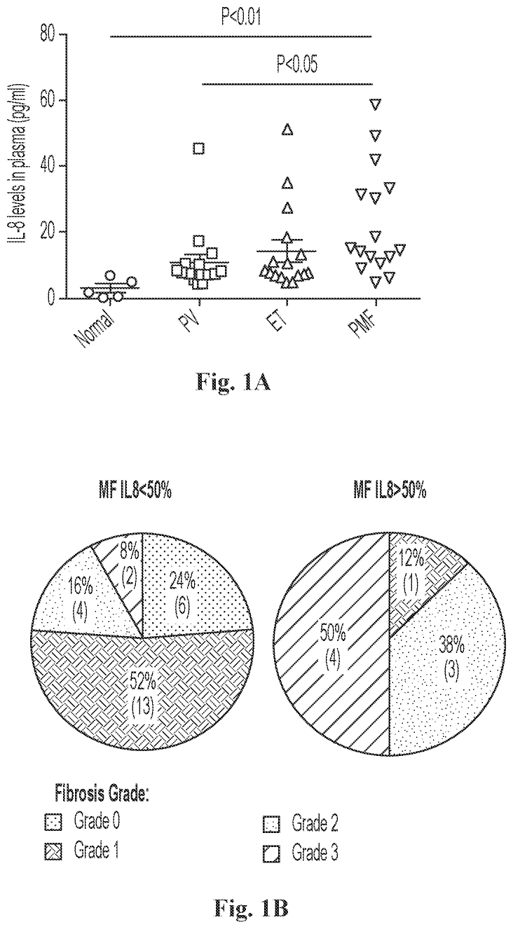

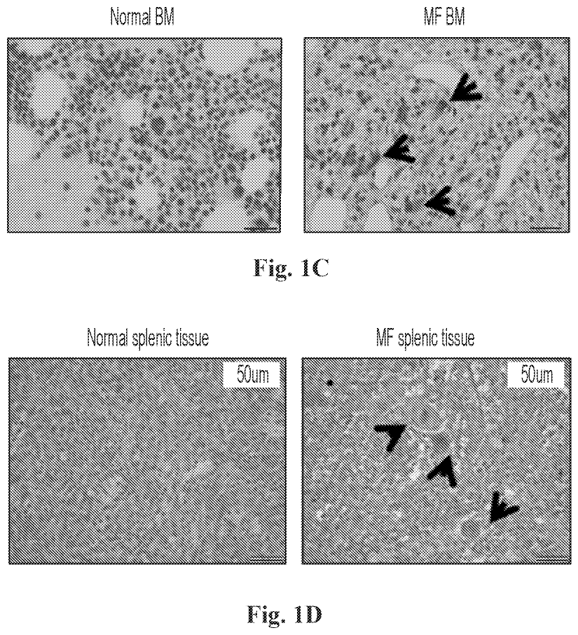

The levels of IL-8 in normal, polycythemia vera (PV), essential thrombocythemia (ET) and MF plasma were assayed with ELISA. MF patient plasma had profoundly higher plasma levels of IL-8 (FIG. 1A).

MF patients with expanded IL-8 secreting clones (defined as >50% of total CD34⁺ cells) had also increased leukocytosis, larger spleen sizes, greater prevalence of constitutional symptoms, and higher-grade reticulin fibrosis in marrow (FIG. 1B) in comparison to MF patients without prevalent IL-8 clones.

Immunohistochemistry confirmed increased IL-8 expression in marrow biopsies from 8/15 MF patients in comparison to 0/4 normal controls (FIG. 1C), and high IL-8 expression was also observed in MF splenic megakaryocytes (MKs) as well as in splenic stromal/endothelial cells not seen in normal spleen (FIG. 1D).

Integrated RNA-Seq and Assay for Transposase-Accessible Chromatin followed by next-generation sequencing (ATAC-Seq) was performed on CD34⁺ cells from myeloproliferative neoplasm (MPN) patients with and without expanded IL-8 secreting clones for gene expression/chromatin accessibility analysis. Analysis of IL-8-high MF patients confirmed up-regulation of IL-8-CXCR2 signaling and enrichment in pro-flammatory pathways (i.e TNFa, NFkB, etc.) by gene set enrichment analysis (GSEA), as well as increased expression/accessibility of pro-inflammatory genes S100A8 and S100A9, previously implicated in fibrosis development.

These data indicate that IL-8 plays an important role in MF disease development.

### Example 2: IL-8 Receptors CXCR1/CXCR2 Play an Important Role in MF

Although IL-8 interacts with many cell surface receptors, the G protein-coupled serpentine receptors CXCR1 and CXCR2 are of primary importance.

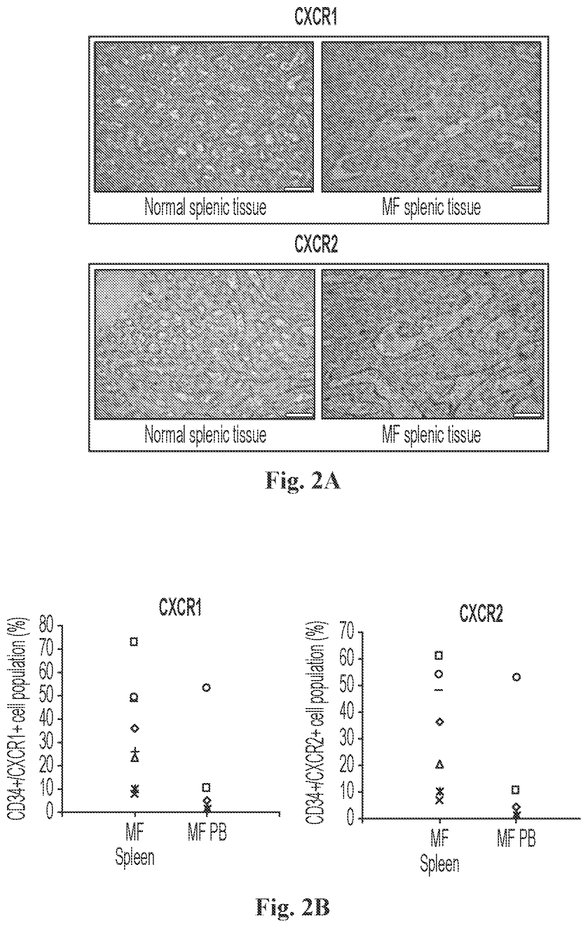

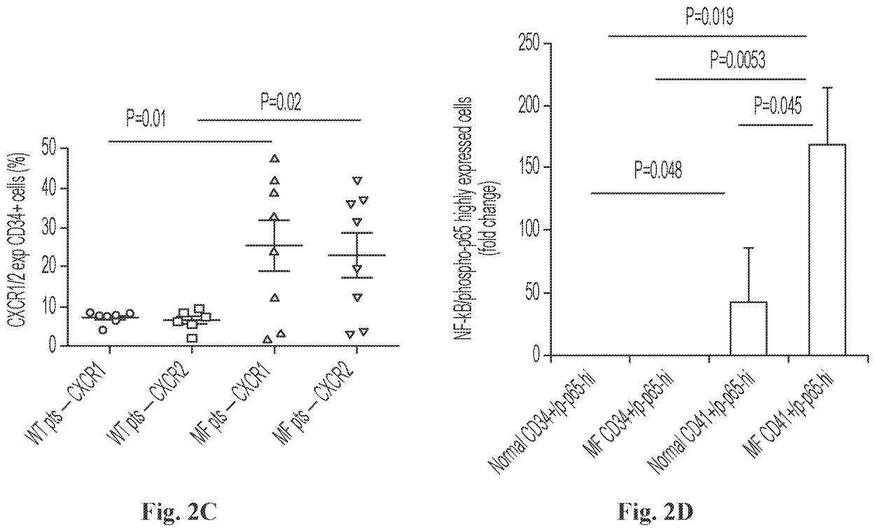

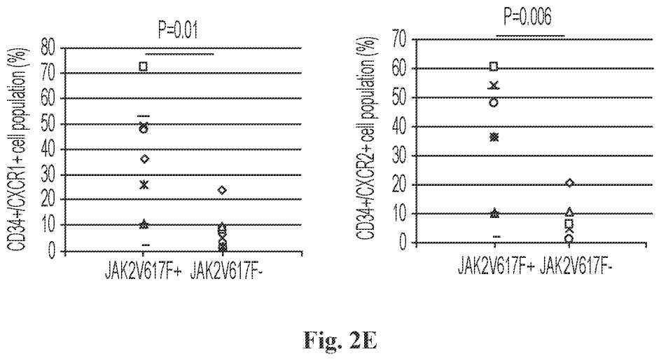

The expression of chemokine receptors CXCR1 and CXCR2 in both normal and MF spleens was determined (FIG. 2A). In both normal and MF spleens, CXCR1 and CXCR2 are expressed to the greatest degree within splenic littoral cells which line the sinusoids within the spleen. These sinusoids are the vessels by which hematopoietic cells return from the spleen to the circulation. Further, the density of littoral cells is diminished within MF spleens as compared to MF spleens. This reduction in numbers of littoral cells may lead to the retention of hematopoietic cells In MF.

Fluorescence-activated cell sorting (FACS) analysis data showed that MF splenic samples contained higher percentage of CD34⁺/CXCR1⁺ and CD34⁺/CXCR2⁺ cells than MF peripheral blood samples (FIG. 2B).

Enhanced surface expression of CXCR2 and its analog CXCR1 in IL-8-high MF CD34⁺ cells as compared to normal cells (FIG. 2C) coincided with enhanced NFkB pathway activity (FIG. 2D).

JAK2 V617F is a mutation frequently found in MF patients. As determined by FACS, a higher fraction of JAK2 V617F positive MF CD34⁺ cells than normal CD34⁺ cells expressed CXCR1 and CXCR2 receptors (p=0.01 and p=0.006, respectively) (FIG. 2E).

### Example 3: Reduction of IL-8 Blocks VEGF, which is Involved in the Development of Splenic Endothelial Cells (EC)/MF HSC Niches

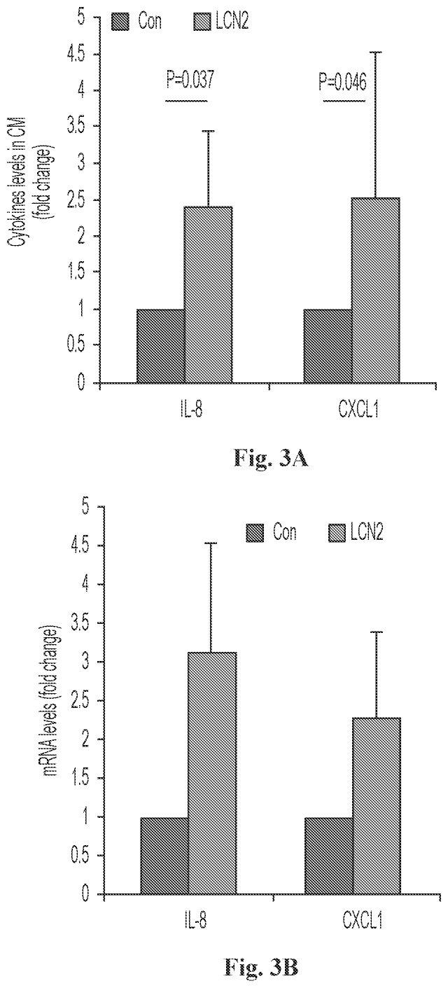

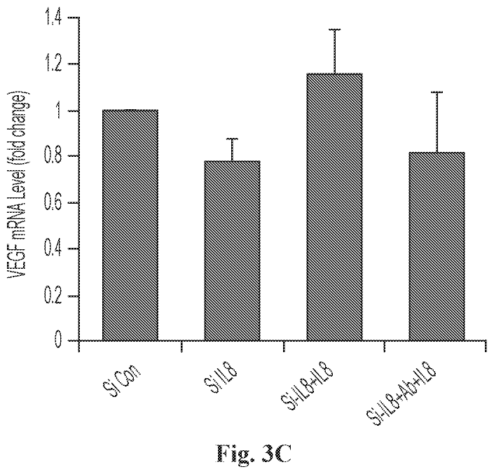

Lipocalin2 (LCN2) is a cytokine produced by MF marrow myeloid cells. Levels of LCN2 are 2-3 fold greater in the circulation of MF patients as compared to PV and ET patients and even higher as compared to healthy controls. Treatment of normal splenic stromal cells with LCN2 led to a significant increase in IL-8 and CXCL1 mRNA and protein levels (FIGS. 3A and 3B). IL-8 and the related chemokine CXCL1 are pro-angiogenic creating a cascade of cytokines (VEGF) contributing to the development of splenic EC/MF HSC niches. Silencing of IL-8 decreased VEGF mRNA expression by spleen stromal cells (FIG. 3C), while addition of IL-8 reversed this effect. The positive impact of IL-8 on VEGF expression was blocked by an IL-8 neutralizing antibody.

In sum, these data identify IL-8 as a pivotal element in MF splenic angiogenesis.

### Example 4: A CXCR1/CXCR2 Inhibitor Reverses Effects of IL-8 on MF CD34⁺ Cells Proliferation and Lineage Differentiation

To evaluate the effects of IL-8 on MF CD34⁺ cells proliferation and lineage differentiation, MF mononuclear cells (MNC cells) were cultured with StemSpan™ Serum-Free Expansion Medium (SFEM) containing 20 ng/ml of stem cell factor (SCF), thrombopoietin (TPO), FL-3L and IL-3 with or without 50 ng/ml of IL-8. The cells were harvested after 3 days of incubation. The proportion of hematopoietic cells belonging to specific lineages were determined using flow cytometry.

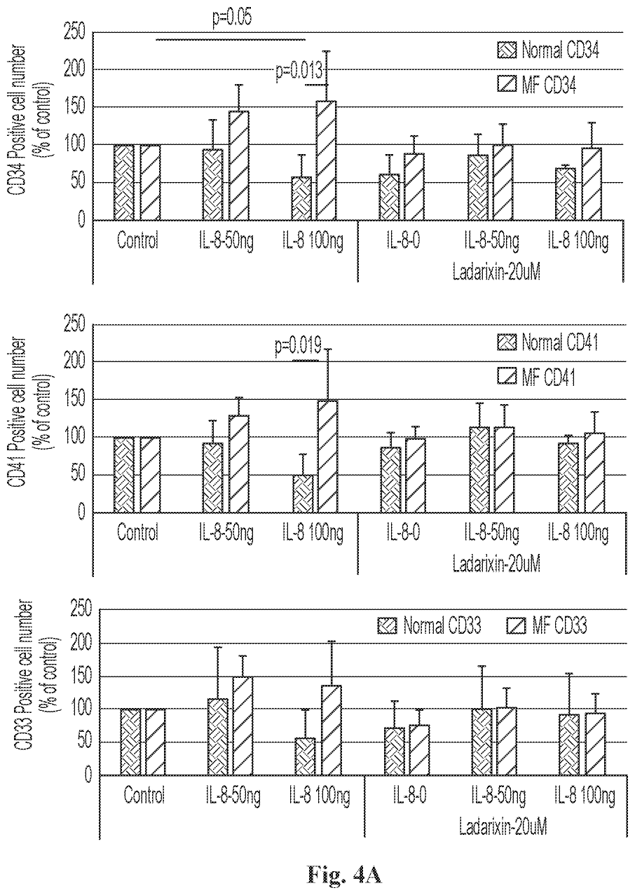

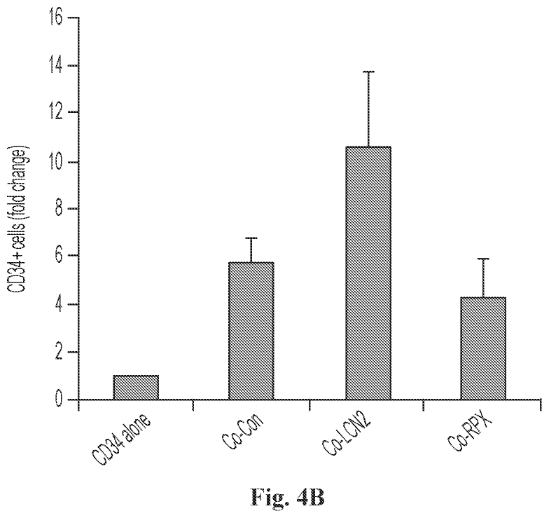

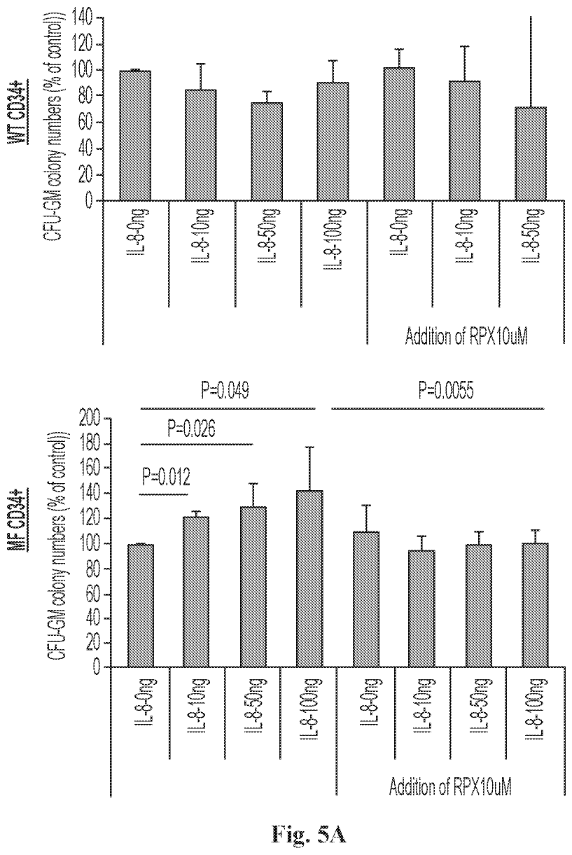

IL-8 increased the percentage of MF CD34⁺, CD41⁺ and CD33⁺ cells but decreased the corresponding cell populations when with normal donor CD34⁺ cells were incubated IL-8. Importantly, treatment with CXCR1/CXCR2 inhibitor ladarixin reversed the effects of IL-8 on both MF and normal cells (FIG. 4A).

Similarly, while treatment with LCN2 enhances endothelial cell (EC)-mediated proliferation of MF cells, treatment with the CXCR1/CXCR2 inhibitor reparixin decreased MF CD34⁺ proliferation when co-cultured with splenic EC (FIG. 4B).

### Example 5: A CXCR1/CXCR2 Inhibitor Reverses Effects of IL-8 on MF CD34+ Cell Colony Formation

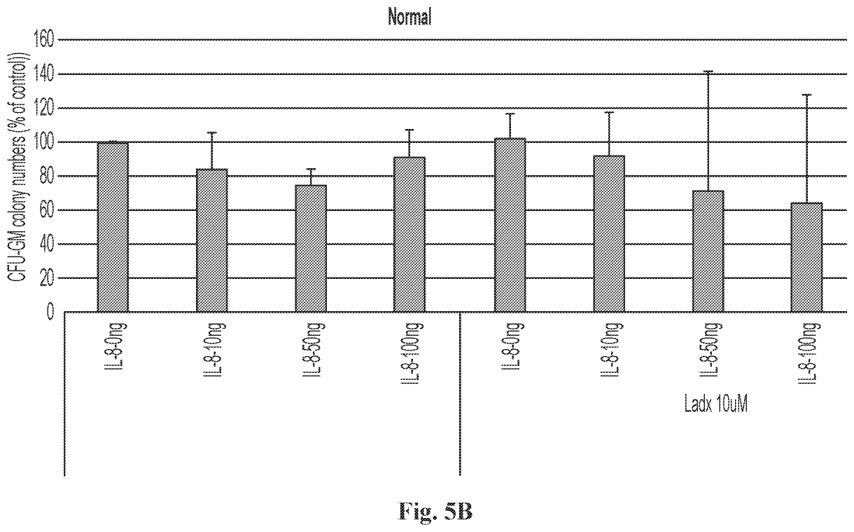

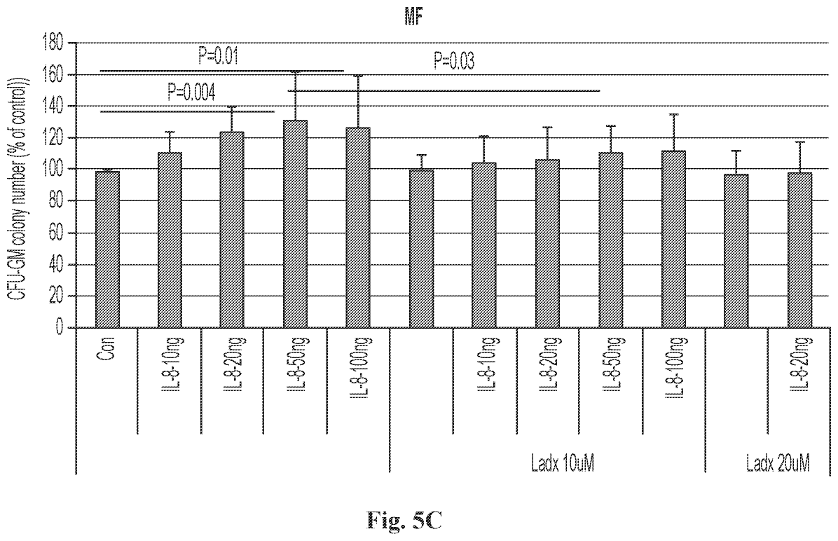

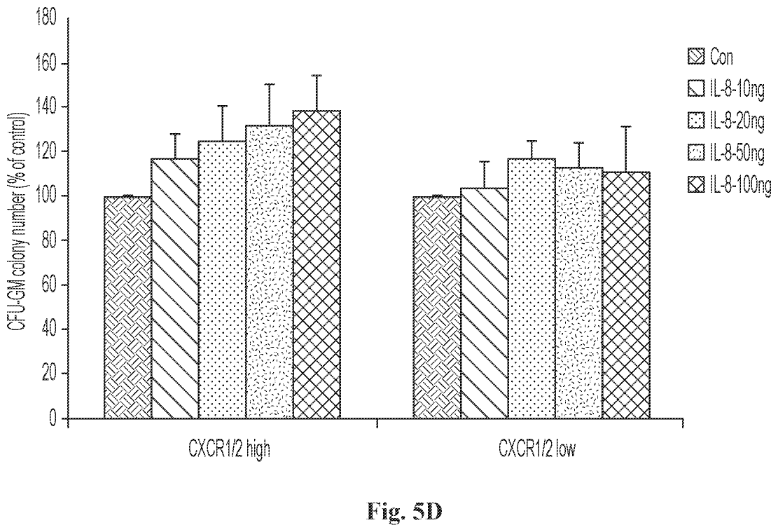

Colony forming assays of cultured MF CD34⁺ cells revealed enhanced colony output when cultured with IL-8 compared to WT CD34⁺ cells—an effect ameliorated by co-treatment with the CXCR1/CXCR2 inhibitor reparixin (FIG. 5A).

Further, the addition of increasing concentrations of IL-8 or CXCR1/CXCR2 inhibitor ladarixin did not affect hematopoietic colony formation by normal CD34⁺ cells. (FIG. 5B). By contrast, IL-8 increased the numbers of MF CFU-GM colonies at doses of 50 ng/ml and 100 ng/ml (p=0.004 and p=0.01, respectively) which was blunted by the addition of CXCR1/CXCR2 inhibitor ladarixin (FIG. 5C).

Similarly, treatment with IL-8 increased CFU-GM colony formation by MF samples with the highest expression of CXCR1/CXCR2 (FIG. 5D) and these effects could be corrected by addition of ladarixin (FIG. 5E).

### Example 6: A CXCR1/CXCR2 Inhibitor Reverses Effects of IL-8 on Malignant HPC

Individual hematopoietic colonies were randomly picked from clonal assays of CD34⁺ cells from 6 MF cases and the JAK2V617F allele status was determined.

IL-8 alone increased the absolute numbers of JAK2V617F+ colonies, and the addition of Ladarixin reduced the numbers of JAK2V617F positive colonies stimulated by IL-8 (Tables 1-3).

TABLE 1
Effects of treatment with IL-8 and CXCR1/CXCR2 inhibitor ladarixin
on the absolute number of hematopoietic colony numbers with a specific
JAK2 genotype generated from MF CD34⁺ cells.

| | | | MF1 | MF1 | MF1 | MF2 | MF2 | MF2 |
|---|---|---|---|---|---|---|---|---|
| Ladarixin | IL-8 | | Heter | Homo | WT | Heter | Homo | WT |
| | | | 41* | 3 | 6 | 0 | 0 | 43 |
| | 10 | ng | 36 | 0 | 18 | 0 | 0 | 44 |
| | 20 | ng | 48 | 0 | 17 | 3 | 0 | 38 |
| | 50 | ng | 52 | 0 | 21 | 7 | 0 | 41 |
| | 100 | ng | 68 | 0 | 0 | 3 | 0 | 52 |
| 10 μM | | | 49 | 4 | 0 | 0 | 0 | 36 |
| 10 μM | 10 | ng | 54 | 8 | 0 | 0 | 0 | 49 |
| 10 μM | 20 | ng | 56 | 4 | 4 | 3 | 0 | 35 |
| 10 μM | 50 | ng | 62 | 5 | 0 | 0 | 0 | 44 |
| 10 μM | 100 | ng | 58 | 8 | 0 | 0 | 0 | 46 |
| 20 μM | | | 43 | 0 | 16 | 0 | 0 | 44 |
| 20 μM | 20 | ng | 49 | 4 | 9 | 0 | 0 | 47 |

Heter = JAK2V617F Heterozygous; Homo = JAK2V617F homozygous; WT = JAK2 Wild type.
*Each number represents the total number of colonies generated from MF 1000 CD34⁺ cells from 6 different patients under the conditions outlined.

TABLE 2
Effects of treatment with IL-8 and CXCR1/CXCR2 inhibitor ladarixin
on the absolute number of hematopoietic colony numbers with a specific
JAK2 genotype generated from MF CD34⁺ cells.

| | | | MF3 | MF3 | MF3 | MF4 | MF4 | MF4 |
|---|---|---|---|---|---|---|---|---|
| Ladarixin | IL-8 | | Heter | Homo | WT | Heter | Homo | Wild |
| | | | 0 | 93 | 28 | 153 | 11 | 0 |
| | 10 | ng | 27 | 80 | 36 | 183 | 12 | 0 |
| | 20 | ng | 79 | 63 | 31 | 171 | 0 | 0 |
| | 50 | ng | 30 | 99 | 30 | 190 | 0 | 0 |
| | 100 | ng | 32 | 95 | 42 | 174 | 12 | 12 |
| 10 μM | | | 13 | 73 | 13 | 163 | 23 | 0 |
| 10 μM | 10 | ng | 110 | 17 | 8 | 137 | 0 | 0 |
| 10 μM | 20 | ng | 17 | 69 | 51 | 113 | 0 | 0 |
| 10 μM | 50 | ng | 50 | 67 | 34 | 137 | 0 | 0 |
| 10 μM | 100 | ng | 0 | 109 | 49 | 112 | 7 | 0 |
| 20 μM | | | 28 | 49 | 35 | 142 | 0 | 0 |
| 20 μM | 20 | ng | 0 | 70 | 53 | 134 | 0 | 0 |

Heter = JAK2V617F Heterozygous; Homo = JAK2V617F homozygous; WT = JAK2 Wild type.

TABLE 3
Effects of treatment with IL-8 and CXCR1/CXCR2 inhibitor ladarixin
on the absolute number of hematopoietic colony numbers with a specific
JAK2 genotype generated from MF CD34⁺ cells.

| | | | MF5 | MF5 | MF5 | MF6 | MF6 | MF6 |
|---|---|---|---|---|---|---|---|---|
| Ladarixin | IL-8 | | Heter | Homo | Wild | Heter | Homo | Wild |
| | | | 0 | 0 | 127 | 0 | 0 | 118 |
| | 10 | ng | 0 | 0 | 124 | 0 | 0 | 115 |
| | 20 | ng | 0 | 0 | 142 | 0 | 0 | 131 |
| | 50 | ng | 0 | 0 | 115 | 8 | 0 | 125 |
| | 100 | ng | 0 | 0 | 115 | 12 | 0 | 87 |
| 10 μM | | | 0 | 0 | 107 | 0 | 0 | 119 |
| 10 μM | 10 | ng | 0 | 0 | 148 | 0 | 0 | 119 |
| 10 μM | 20 | ng | 0 | 0 | 138 | 0 | 0 | 121 |
| 10 μM | 50 | ng | 0 | 0 | 151 | 0 | 0 | 122 |
| 10 μM | 100 | ng | 0 | 0 | 144 | 0 | 0 | 119 |
| 20 μM | | | 0 | 0 | 141 | 0 | 0 | 105 |
| 20 μM | 20 | ng | 0 | 0 | 131 | 0 | 0 | 106 |

Heter = JAK2V617F Heterozygous; Homo = JAK2V617F homozygous; WT = JAK2 Wild type.

These data indicate that IL-8 can differentially affect classes of MF hematopoietic progenitor cells based on their mutational status and that these effects can be reversed by pharmacologically antagonizing CXCR1/2.

### Example 7: A CXCR1/CXCR2 Inhibitor Reverses Effects of IL-8 on Micro-Environmental Cells

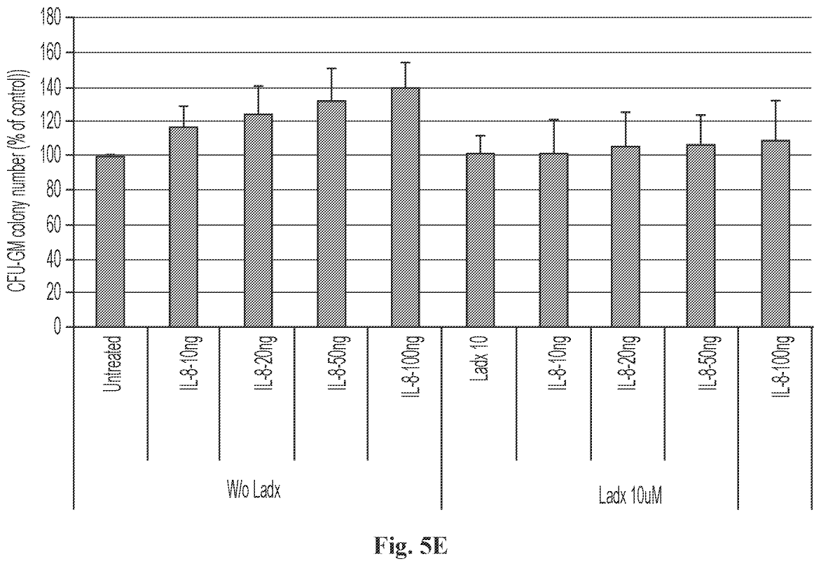

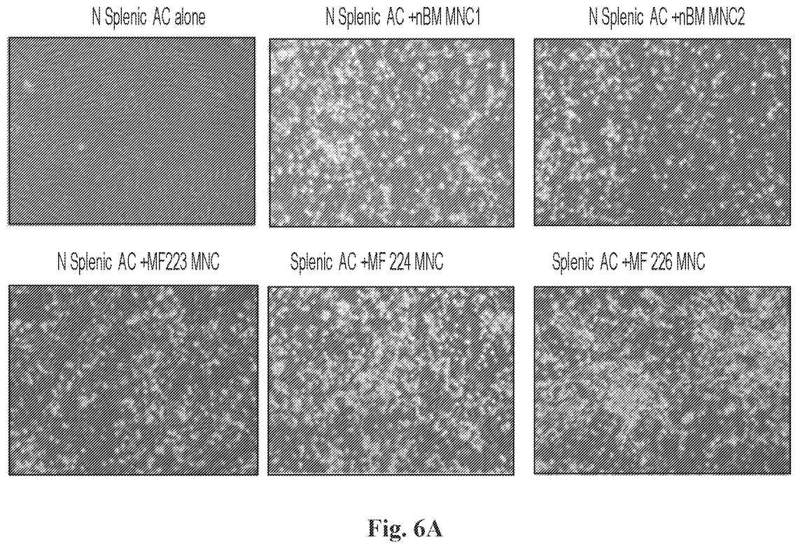

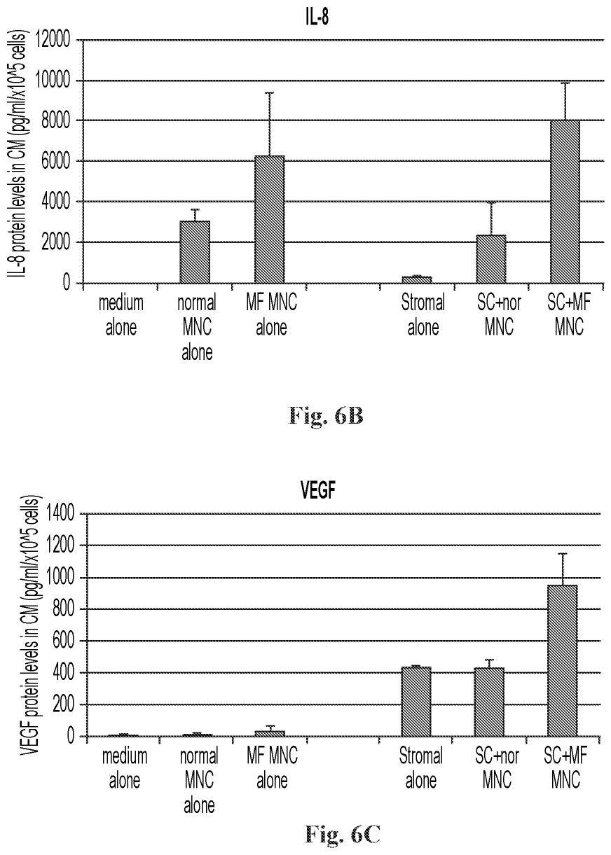

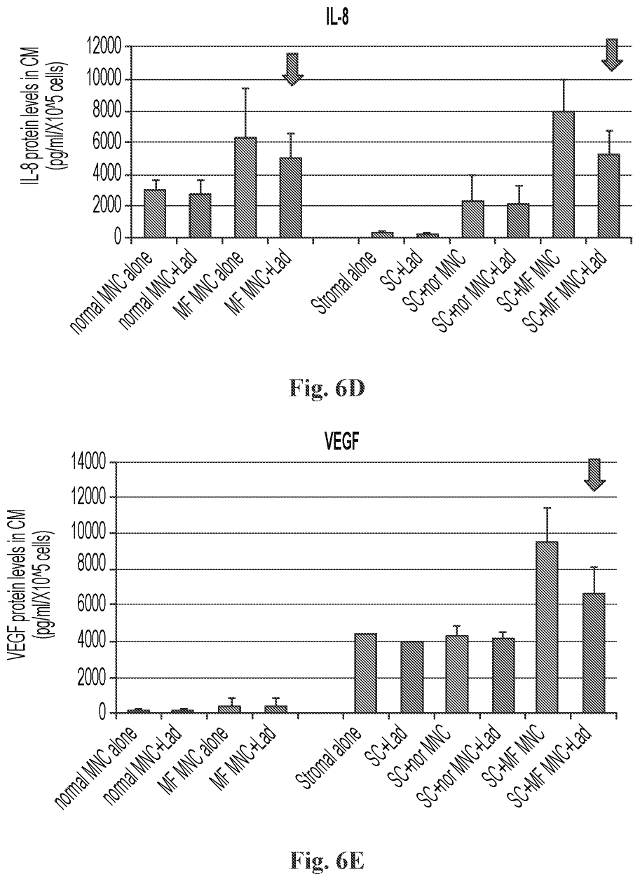

IL-8 not only targets hematopoietic cells, but also affects micro-environmental cells such as marrow and spleen endothelial and stromal cells. MF MNC cells co-cultured with splenic stromal cells altered morphological in the stromal cells (FIG. 6A).

ELISA analysis showed that both IL-8 and VEGF levels were increased in conditioned media from co cultures of MF and normal MNCs with normal splenic stromal cells (FIGS. 6B and 6C).

Addition of Ladarixin decreased IL-8 and VEGF levels in these co-cultures (FIGS. 6D and 6E).

1. A method of treating myelofibrosis (MF), the method comprising administering to a subject in need thereof an effective amount of a CXCR1/CXCR2 inhibitor.

2. A method of decreasing bone marrow fibrosis, spleen volume, plasma VEGF levels, bone marrow microvessel density, bone marrow megakaryocyte number, number of IL-8 secreting clones, and/or number of peripheral blood CD34⁺ cells in a subject, the method comprising administering to a subject in need thereof an effective amount of a CXCR1/CXCR2 inhibitor.

3-5. (canceled)

6. The method of claim 2, wherein the subject has myelofibrosis.

7. The method of claim 1, wherein the subject is unresponsive to or ineligible for janus kinase inhibitor (JAKi) treatment.

8. The method of claim 1, wherein the CXCR1/CXCR2 inhibitor is administered as a pharmaceutical composition comprising the CXCR1/CXCR2 inhibitor and one or more pharmaceutically acceptable excipients.

9. The method of claim 1, wherein the CXCR1/CXCR2 inhibitor is a compound of formula (I) 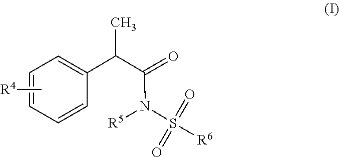
or a pharmaceutically acceptable salt thereof,
wherein
R⁴ is linear or branched C₁-C₆ alkyl, benzoyl, phenoxy, trifluoromethanesulfonyloxy; preferably it is selected from benzoyl, isobutyl and trifluoromethanesulfonyloxy. Also, according to a preferred embodiment R⁴ is in position 3 or 4 on the phenyl ring, more preferably it is 3-benzoyl, 4-isobutyl or 4-trifluoromethanesulfonyloxy.
R⁵ is H or linear or branched C₁-C₃ alkyl, preferably it is H.
R⁶ is linear or branched C₁-C₆ alkyl or halo C₁-C₃ alkyl, preferably it is CH₃ or trifluoromethyl.

10. The method of claim 1, wherein the CXCR1/CXCR2 inhibitor is a compound of formula (II) 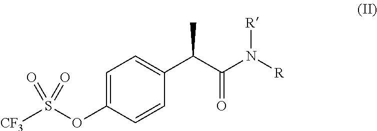
or a pharmaceutically acceptable salts thereof,
wherein
R′ is hydrogen;
R is a residue of formula SO2Ra wherein Ra is C₁-C₆ alkyl or halo C₁-C₃ alkyl, preferably it is CH₃ or trifluoromethyl.

11. The method of claim 1, wherein the CXCR1/CXCR2 inhibitor is a compound of formula (V) 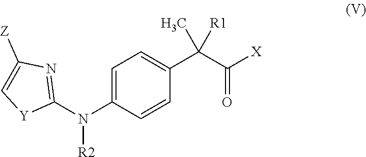
wherein
R1 is hydrogen;
X is OH;
R2 is hydrogen or linear C₁-C₄ alkyl;
Y is a heteroatom selected from S, O and N;
Z is selected from linear or branched C₁-C₄ alkyl, linear or branched C₁-C₄ alkoxy, halo C₁-C₃ alkyl and halo C₁-C₃ alkoxy.

12. The method of claim 1, wherein the CXCR1/CXCR2 inhibitor is R(-)-2-[(4′-trifluoromethanesulfonyloxy)phenyl]-N-methanesulfonyl propionamide or its sodium salt.

13. (canceled)

14. The method of claim 1, wherein the CXCR1/CXCR2 inhibitor is R-(-)-2-(4-isobutylphenyl)propionyl methansulfonamide or its lysine salt.

15. (canceled)

16. The method of claim 2, wherein the subject is unresponsive to or ineligible for janus kinase inhibitor (JAKi) treatment.

17. The method of claim 2, wherein the CXCR1/CXCR2 inhibitor is administered as a pharmaceutical composition comprising the CXCR1/CXCR2 inhibitor and one or more pharmaceutically acceptable excipients.

18. The method of claim 2, wherein the CXCR1/CXCR2 inhibitor is a compound of formula (I) 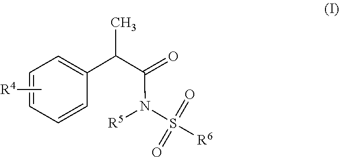
or a pharmaceutically acceptable salt thereof,
wherein
R⁴ is linear or branched C₁-C₆ alkyl, benzoyl, phenoxy, trifluoromethanesulfonyloxy; preferably it is selected from benzoyl, isobutyl and trifluoromethanesulfonyloxy. Also, according to a preferred embodiment R⁴ is in position 3 or 4 on the phenyl ring, more preferably it is 3-benzoyl, 4-isobutyl or 4-trifluoromethanesulfonyloxy.
R⁵ is H or linear or branched C₁-C₃ alkyl, preferably it is H.
R⁶ is linear or branched C₁-C₆ alkyl or halo C₁-C₃ alkyl, preferably it is CH₃ or trifluoromethyl.

19. The method of claim 2, wherein the CXCR1/CXCR2 inhibitor is a compound of formula (II) 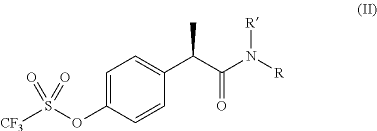
or a pharmaceutically acceptable salts thereof,
wherein
R′ is hydrogen;
R is a residue of formula SO2Ra wherein Ra is C₁-C₆ alkyl or halo C₁-C₃ alkyl, preferably it is CH₃ or trifluoromethyl.

20. The method of claim 2, wherein the CXCR1/CXCR2 inhibitor is a compound of formula (V) 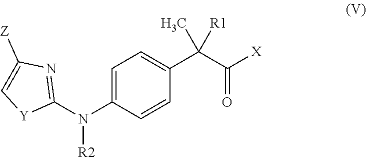
wherein
R1 is hydrogen;
X is OH;
R2 is hydrogen or linear C₁-C₄ alkyl;
Y is a heteroatom selected from S, O and N;
Z is selected from linear or branched C₁-C₄ alkyl, linear or branched C₁-C₄ alkoxy, halo C₁-C₃ alkyl and halo C₁-C₃ alkoxy.

21. The method of claim 2, wherein the CXCR1/CXCR2 inhibitor is R(-)-2-[(4′-trifluoromethanesulfonyloxy)phenyl]-N-methanesulfonyl propionamide or its sodium salt.

22. The method of claim 2, wherein the CXCR1/CXCR2 inhibitor is R-(-)-2-(4-isobutylphenyl)propionyl methansulfonamide or its lysine salt.

23. A method of treating myelofibrosis (MF), the method comprising administering to a subject in need thereof an effective amount of a CXCR1/CXCR2 inhibitor.

24. A method of decreasing bone marrow fibrosis, spleen volume, plasma VEGF levels, bone marrow microvessel density, bone marrow megakaryocyte number, number of IL-8 secreting clones, and/or number of peripheral blood CD34⁺ cells in a subject, the method comprising administering to a subject in need thereof an effective amount of a CXCR1/CXCR2 inhibitor.
